(19) United States
(12) Patent Application Publication
(10) Pub. No.: US 2017/0074873 A1
(43) Pub. Date: Mar. 16, 2017

(54) IMMUNOASSAY KIT

(71) Applicant: REGA BIOTECHNOLOGY INC., New Taipei City (TW)

(72) Inventor: SU-YU LIN, New Taipei City (TW)

(21) Appl. No.: 15/259,173

(22) Filed: Sep. 8, 2016

(30) Foreign Application Priority Data
Sep. 11, 2015 (CN) 201510574230.3

(51) Int. Cl. G01N 33/543; G01N 21/78

(52) U.S. Cl. CPC G01N 33/54366; G01N 33/54306; G01N 21/78; G01N 2021/7759

(57) ABSTRACT

Differing from conventional lateral flow immunoassay test strips, the present invention provides an immunoassay kit comprising a release strip and a reaction strip. When using this immunoassay kit to complete a competitive-type immunochromatographic test or a sandwich-type immunochromatographic test for a test sample, it needs to firstly put the release strip into the test sample for releasing a specific antibody (or antigen) conjugating with a marker into the test sample, so as to make the first antibody connect to a target object in the test sample; after that, the reaction strip is eventually put into the test sample after the release strip is removed out of the test sample. Therefore, the qualitative analysis result of the competitive/sandwich-type immunochromatographic test can be easily obtained by determining whether the test line shows the color reaction or not.

### BACKGROUND OF THE INVENTION

1. Field of the Invention

The present invention relates to the technology field of immunochromatographic test, and more particularly to an immunoassay kit consisting of a releasing strip and a reaction strip.

2. Description of the Prior Art

Recently, immunochromatographic test gets more and more attention because of having the advantages of simple structure and low manufacturing cost. For the maximum sensitivity of the immunochromatographic test being up to 10⁻¹² g/mL, the immunochromatographic test has been broadly applied in following fields:
(1) Clinical field, where the immunochromatographic test is used for carrying out the detections of allergies, drugs, infectious diseases, endocrine diseases, tumor markers, etc.
(2) Agricultural field, where the immunochromatographic test is used for verifying food safety and plant diseases.
(3) Environment field, where the immunochromatographic test is used for ascertaining biological and environmental pollutions.
(4) Veterinary field, where the immunochromatographic test is used for verifying animal diseases.

Lateral flow immunoassay is one kind of the immunochromatographic test assay, and commonly used for completing the detections of various liquid samples. Please refer to FIG. 1, which illustrates a schematic structural view of a conventional lateral flow immunoassay test strip. As shown in FIG. 1, the conventional lateral flow immunoassay test strip 1′ consists of: a substrate 11′, a membrane 12′, a conjugation pad 13′, a sample pad 14′, and an absorption pad 15′.

In the lateral flow immunoassay test strip 1′ shown by FIG. 1, the membrane 12′ is disposed on the substrate 11′ and used for conjugating one test sample with an antibody, an antigen, a DNA, or a RNA. Particularly, the compositions for forming a test line 12T′ and a control line 12C′ will be different according to one specific implemented type of the immunochromatographic test assay carried out on the lateral flow immunoassay test strip 1′, such as competitive type immunochromatographic test assay and sandwich type immunochromatographic test assay. When the sandwich type immunochromatographic test assay is implemented on the lateral flow immunoassay test strip 1′, the test line 12T′ is formed by an antibody 2 (Ab2) and the control line 12C′ is formed by a second antibody (which is anti-Ab1); moreover, a first antibody (Ab1) conjugating with a maker (CGC-Ab1) is further disposed in the conjugation pad 13′, wherein the maker is colloidal gold and the CGC means colloidal gold conjugate.

Therefore, after a specific sample is dropped onto the sample pad 14′, the antigen in the sample would move toward the conjugation pad 13′ under the capillarity effect, so as to conjugated with the antibody 1 having the marker (CGC-Ab1). Next, the antigen and the first antibody (Ab1) connecting with one terminal of the antigen would continuously move toward the membrane 12′; meanwhile, the antibody 2 (Ab2) on the test line 12T′ would conjugate to the other terminal of the antigen, such that the test line 12T′ would show a color reaction. Furthermore, the antigen and the first antibody (Ab1) which does not conjugate with the antibody 2 (Ab2) on the test line 12T′ would continuously move toward the control line 12C′, so as to connect with the second antibody for making the control line 12C′ show a color reaction. Herein, it needs to further explain that, the qualitative analysis result of the sandwich type immunochromatographic test can be obtained by determining whether the test line 12T′ does show the color reaction or not. To put that simply, the qualitative analysis result of the sandwich type immunochromatographic test is positive when the test line 12T′ show the color reaction, and the opposite is negative.

On the contrary, when the competitive type immunochromatographic test assay is implemented on the lateral flow immunoassay test strip 1′, the test line 12T′ is formed by a competing antigen (Ag) conjugate with carrier protein (carrier-Ag) and the control line 12C′ is also formed by the second antibody (anti-Ab1); in addition, the first antibody (Ab1) conjugating with a maker (CGC-Ab1) is further disposed in the conjugation pad 13′. Therefore, after a specific sample is dropped onto the sample pad 14′, the antigen in the sample would move toward the conjugation pad 13′ under the capillarity effect, so as to conjugated with the CGC-Ab1. Next, the antigen and the CGC-Ab1 connecting with one terminal of the antigen (Ag-Ab1-CGC) would continuously move to the membrane 12′; meanwhile, the Carrier-Ag on the test line 12T′ would compete with the CGC-Ab1. Furthermore, the free Ag-Ab1-CGC continuously moves toward the control line 12C′, so as to conjugate with the second antibody (anti-Ab1) for making the control line 12C′ show a color reaction. Herein, it needs to further explain that, the qualitative analysis result of the competitive type immunochromatographic test can be obtained by determining whether the test line 12T′ does show the color reaction or not. To put that simply, the qualitative analysis result of the competitive type immunochromatographic test is negative when the test line 12T′ show the color reaction, and the opposite is positive.

The commonly-used markers include latex and colloidal gold. Moreover, in order to facilitate the antibody having the marker be able to easily move from the conjugation pad 13′ to the test line 12T′ and/or the control line 12C′ on the membrane 12′, manufacturers of the immunoassay test strips particularly fabricate the conjugation pad 13′ by microporous materials. Therefore, the sensitivity of the immunoassay test strips is effectively enhanced.

However, in spite of that, the conventional lateral flow immunoassay test strip 1′ has been found following drawbacks in practical use:

(1) the use of the microporous materials cause the manufacturing cost of the immunoassay test strips be increase; moreover, the immunoassay test strips having the microporous materials must be stored under a low-temperature environment of 4° C., and the maximum storage life of the microporous materials is merely 1 year.

(2) because the commercial (competitive type) lateral flow immunoassay test strip 1′ cannot effectively inhibit the test line 12T′ from showing the color reaction, it needs to further use colorimetry to verify the chrominance of the test line 12T′ and the control line 12C′. However, the maximum sensitivity of the aflatoxin M1 colorimetry verification can only up to 0.5 ppb.

Accordingly, in view of the conventional lateral flow immunoassay test strip include many drawbacks, the inventor of the present application has made great efforts to make inventive research thereon and eventually provided an immunoassay kit.

### SUMMARY OF THE INVENTION

The primary objective of the present invention is to provide an immunoassay kit. Differing from conventional lateral flow immunoassay test strips, this novel immunoassay kit comprises a release strip and a reaction strip. When using this immunoassay kit to complete a competitive-type immunochromatographic test or a sandwich-type immunochromatographic test for a test sample, it needs to firstly put the release strip into the test sample for releasing a specific antibody (or antigen) conjugating with a marker into the test sample, so as to make the first antibody connect to a target object in the test sample; after that, the reaction strip is eventually put into the test sample after the release strip is removed out of the test sample. Therefore, the qualitative analysis result of the competitive/sandwich-type immunochromatographic test can be easily obtained by determining whether the test line shows the color reaction or not.

Accordingly, in order to achieve the primary objective of the present invention, the inventor of the present invention provides a first embodiment for the immunoassay kit, used for detecting a target object from a test sample by way of a competitive-type immunochromatographic test, mainly comprising: a release strip and a reaction strip. In which, the release strip is provided with a first antibody (Ab1) conjugating with a marker (CGC-Ab1) thereon, and the reaction strip is provided with a membrane having a control line and a test line thereon. Moreover, the control line is formed by a second antibody (anti-Ab1), and the test line is formed by a conjugate consisting of a specific antigen and a biomolecule (e.g., Carrier-Ag), wherein the first antibody is used for connecting to the target object, and the said specific antigen is the antigen of the target object. When completing the competitive-type immunochromatographic test by using the immunoassay kit, the release strip needs to be firstly put into the test sample for releasing the first antibody conjugating with the marker (CGC-Ab1) into the test sample, and the reaction strip is eventually put into the test sample after the release strip is removed out of the test sample. Therefore, only the control line on the reaction strip would show a color reaction when the target object is included by the test sample.

Moreover, for achieving the primary objective of the present invention, the inventor of the present invention provides a second embodiment for the immunoassay kit, used for detecting a target object from a test sample by way of a sandwich-type immunochromatographic test, mainly comprising a release strip and a reaction strip. In which, the release strip is provided with a first antibody (Ab1) conjugating with a marker (CGC-Ab1) thereon, and the first antibody (Ab1) is used for connecting to the target object (e.g., Antigen (Ag)). In addition, the reaction strip is provided with a membrane having a control line and a test line thereon, wherein the control line is formed by a second antibody (anti-Ab1), and the test line is formed by an antibody2 (Ab2); moreover, the first antibody (Ab1) and antibody2 (Ab2) are used for connecting to the target object. when completing the sandwich-type immunochromatographic test by using the immunoassay kit, the release strip needs to be firstly put into the test sample for releasing the first antibody conjugating with the marker (CGC-Ab1) into the test sample, and the reaction strip is eventually put into the test sample after the release strip is removed out of the test sample. Therefore, the control line and the test line on the reaction strip would simultaneously show a color reaction when the target object is included by the test sample.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a schematic structural view of a conventional lateral flow immunoassay test strip;

FIG. 2 shows a schematic structural view of a first exemplary embodiment of an immunoassay kit according to the present invention;

FIG. 3 shows an image view of multi reaction strips of the immunoassay kit;

FIG. 4 shows image views of multi mono-strip-type aflatoxin kits provided by manufacturer A;

FIG. 5 shows image views of multi mono-strip-type aflatoxin kits provided by manufacturer B;

FIG. 6 shows a schematic structural view of a second exemplary embodiment of the immunoassay kit according to the present invention; and

FIG. 7 shows a schematic structural view of a third exemplary embodiment of the immunoassay kit according to the present invention.

### DETAILED DESCRIPTION OF THE PREFERRED EMBODIMENTS

To more clearly describe an immunoassay kit according to the present invention, embodiments of the present invention will be described in detail with reference to the attached drawings hereinafter.

Aflatoxin is the most common toxic pollutants existing in mildewed crops, such as Corn, peanut, rice, wheat, and nuts. Aflatoxin is one kind of the carcinogenic substances belong to Category 1 classified by International agency for research on cancer (IARC). According to the classification of IARC, aflatoxin can be further divided into 4 types, including: aflatoxin B1, aflatoxin B2, aflatoxin G1, and aflatoxinG2, wherein the most common type is aflatoxin B1. After a human (or animal) takes the food (or feed) polluted by aflatoxin B1, the aflatoxin B1 would be metabolized to aflatoxin M1 in human body so as to damage the liver. So that, if a mammal takes the food (or feed) polluted by aflatoxin B1, the milk lactated by the mammal will include the aflatoxin M1. For above reason, U.S. Food and Drug Administration (FDA) especially limits that the concentration of the aflatoxin M1 containing by milk or dairy products cannot exceed 0.5 ppb.

The present invention provides an immunoassay kit for detecting aflatoxin M1 or wheat from a test sample by way of immunochromatographic test. Herein, the test sample can be urine, tissue fluid, gravy, soup, liquid dairy product, liquid sample obtained by dissolving solid sample in a solvent, and an extract of the aforesaid liquid sample, wherein the solvent can be water or sample buffer, and the sample buffer can be a phosphate solution or a high-concentration phosphate solution.

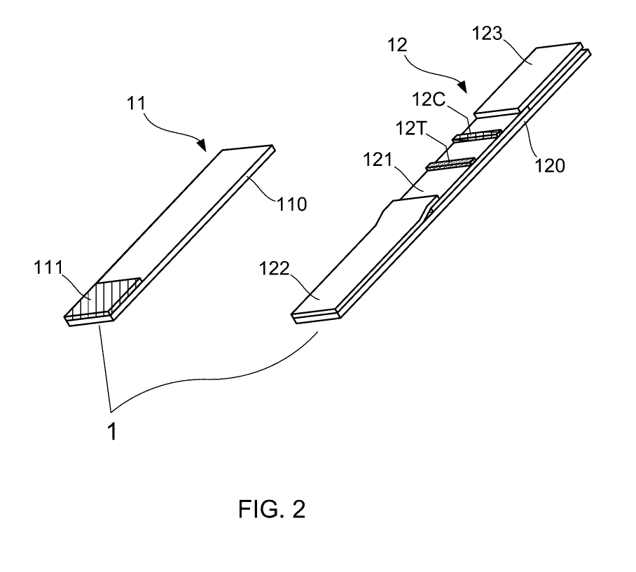

please refer to FIG. 2, which illustrates a schematic structural view of a first exemplary embodiment of an immunoassay kit according to the present invention. As shown in FIG. 2, the immunoassay kit 1 provides by the present invention consists of: a release strip 11 and a reaction strip 12. The main frame of the release strip 11 is a substrate 110, and the front end of the substrate 110 is disposed with a conjugation pad 111 made of a microporous material.

As shown in FIG. 2, the main frame of the release strip 12 is also a substrate 120 provided with a membrane 121 thereon. Besides, a control line 12C and a test line 12T are disposed on the membrane 121. Moreover, the membrane 121 is further provided with a sample pad 122 and an absorption pad 123 thereon, wherein the sample pad 122 and the absorption pad 123 are connected to the front end and rear end of the membrane 121, respectively. In the present invention the membrane is made of nitrocellulos (NC), polyvinylidene difluoride (PVDF), or nylon, and the sample pad 122 is made of polyethylene (PE) or glass fiber.

Therefore, the foundational structure of the immunoassay kit 1 shown in FIG. 2 are clearly introduced. Next, in follows, a first exemplary embodiment of the immunoassay kit 1 will be introduced based on the foundational structure.

### First Embodiment

The first embodiment of the immunoassay kit 1 is design for carrying out a competitive-type immunochromatographic test. As shown in FIG. 2, a first antibody (Ab1) conjugating with a marker (CGC-Ab1) is disposed on the conjugation pad 111 of the release strip 11 in the first embodiment of the immunoassay kit 1, wherein the marker can be a colloidal gold and the CGC means a colloidal gold conjugate. Moreover, the said first antibody (Ab1) can be rabbit anti-aflatoxin antibody or mouse anti-aflatoxin antibody. Moreover, the present invention does not limit the types of the marker, such that the marker can also be colloidal goldcolloidal selenium, latex, nano silver, or nano carbon. Different from the conjugation pad 111, the control line 12C is formed by a second antibody (anti-Ab1), such as goat anti-rabbit immunoglobulin G (IgG), rabbit anti-mouse immunoglobulin G, goat anti-mouse immunoglobulin G and so on. In addition, the test line 12T is formed by a conjugate consisting of a specific antigen (Ag) and a biomolecule (e.g., Carrier-Ag like Ag-OVA, Ag-CMO, or Ag-BSA), wherein the said specific antigen is the antigen of a target object included by the test sample, and the biomolecule can be bovine serum albumin (BSA), ovalbumin (OVA), or choline monooxygenase (CMO). Besides, both the first antibody and the second antibody can be a monoclonal antibody or a polyclonal antibody.

Herein, it needs to further explain that, the purpose of the related design for the first embodiment is to make the immunoassay kit 1 detect the aflatoxin M1 existing in the test sample effectively and rapidly. However, the above described related design for the first embodiment cannot be used for limiting any practicable embodiment of the immunoassay kit 1 proposed by the present invention. The primary technology feature of the present invention is that the immunoassay kit 1 consists of one release strip 11 and one reaction strip 12. So that, based on this technology feature, it reasonably believes that, the person ordinarily skilled in immunoassay kit art can based on their engineering experiences to properly change the kinds of the antibody and/or antigen, so as to facilitate the immunoassay kit 1 be able to detect any one kind of target objective, such as antigen, virus, protein, DNA, RNA, small molecule and so on. In which, the said molecule can be toxin, antibiotic, drug, or related derivatives. Moreover, the said toxin is like aflatoxin M1, aflatoxin B1, or aflatoxins. In addition, the said antibiotic may be β-lactam, for example, penicillin, penicillin derivatives, cephalosporin, carbapenem, and carbapenem inhibitors.

The immunoassay kit 1 provided by the present invention is suitable for detecting the target object from a liquid sample under immunochromatographic test, wherein the liquid sample can be a raw milk, a sterilized whole milk, a low-fat milk, a drink including dairy compositions, or a derivative product of milk. Herein, it needs to further explain that, the aforesaid solid sample can be milk powder, reconcile milk powder, or feed.

In order to verify the practicability of the first embodiment of the immunoassay kit 1, the inventors of the immunoassay kit 1 has complete related experiments for prove that.

### Experiment 1

### Treating a Competitive-Type Immunochromatographic Test to a Liquid Dairy Product

Liquid dairy product is taken as the test sample in experiment 1, wherein the said liquid dairy product is whole milk, milk make from commercial powdered milk based on the manufacturer's suggestion, and commercial yogurt. Before executing the competitive-type immunochromatographic test, the milk make from commercial powdered milk needs to be cooled down to room temperature in advance. Moreover, because yogurt is a thick liquid sample, which needs to be treated with an extracting process, consisting of following steps:
Step (A): adding 16 mL yogurt into a first tube including 16 mL Ethyl acetate, and then slightly overturn the first tube up and down for 30 seconds;
Step (B): using a centrifuge to treat the first tube with a centrifugal process for 10 minutes;
Step (C): moving the supernatant in the first tube to a second tube, and blowing the supernatant in the second tube to dry by using nitrogen gas;
Step (D): sequentially adding 2 mL Hexane and 1 mL sample buffer (i.e., Phosphate-buffered saline (PBS)) into the second tube, and then evenly shaking the second tube for 30 seconds;
Step (E): using the centrifuge to treat the second tube with the same centrifugal process for 10 minutes; and
Step (F): removing the supernatant in the second tube, and then an extract of the liquid sample is obtained.

After the liquid sample is cooled down to room temperature, 3 drops of the liquid sample are added into a small tube by using a first dropper; and then, 3 drops of dilution buffer (i.e., phosphate solution) are added into the small tube by using a second dropper. Next, after letting the front end (i.e., the conjugation pad 111) of the release strip 11 be put into the small tube, the small tube is slightly rotated for 30 seconds, so as to make the first antibody (Ab1) conjugating with the marker be released into the sample solution in the small tube from conjugation pad 111. Meanwhile, the sample solution would show a specific color, e.g., light pink. After statically standing the small tube for 3 minutes, the aflatoxin M1 included by the sample solution would connect to the first antibody (Ab1) (i.e., rabbit anti-aflatoxin antibody or mouse anti-aflatoxin antibody).

Subsequently, after removing the release strip 11 from the small tube, the front end of the reaction strip 12 is put into the small tube, so as to make the sample pad 122 be soaked in the sample solution for 10-15 minutes. Meanwhile, the conjugate consisting of a specific antigen and a bovine serum albumin (BSA) fixed on the test line 12T of the reaction strip 12 and the second antibody (i.e., Immunoglobulin G (IgG)) fixed on the control line 12C would compete to each other for the aflatoxin M1 in the sample solution.

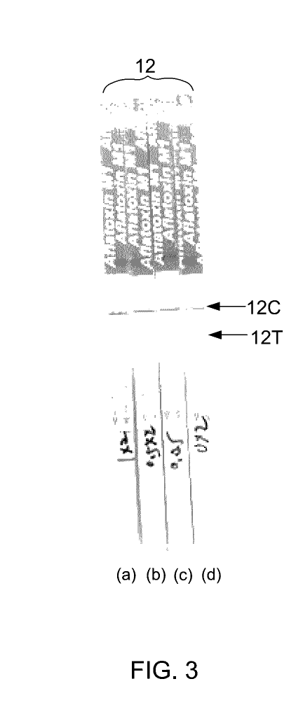

Please refer to FIG. 3, where an image view of multi reaction strips of the immunoassay kit is shown. For the conjugate fixed on the test line 12T be used to competing for the aflatoxin M1 in the sample solution with the second antibody, there is only that the control line 12C show a color reaction when the aflatoxin M1 is indeed included by the sample solution. Briefly, there is only that the control line 12C would show the color reaction when the qualitative analysis result of the competitive-type immunochromatographic test is positive. On the contrary, when the sample solution does not include aflatoxin M1 or includes aflatoxin M1 with low-concentration, the antigen fixed on the test line 12T would simultaneously connect to the aflatoxin M1; meanwhile, both the test line 12T and the control line 12C show the color reaction. Furthermore, a comparison experiment between commercial mono-strip-type aflatoxin kits (i.e., one strip) and the immunoassay kit (i.e., two strips) of the present invention has been made by the inventors, and related experimental data are therefore recorded in following Table 1.

TABLE 1

| | color verification | color verification | color verification |
|---|---|---|---|
| | value obtained | value obtained | value obtained |
| | by using | by using | by using |
| | chromatometry | chromatometry | chromatometry |
| | the immunoassay | mono-strip-type | mono-strip-type |
| concen- | kit 1 provided by | aflatoxin kits | aflatoxin kits |
| tration | the present | provided by | provided by |
| of aflatoxin | invention | manufacturer A | manufacturer B |
| M1 | T value | T/C value | T/C value |
| 5.0 ppb | 1.86 | | |
| 1.0 ppb | 3.55 | | |
| 0.5 ppb | 3.86 | 7.28/49.5 (T < C) | |
| 0.2 ppb | | 45.92/47.12 | 15.86/34.37 (T < C) |
| 0.1 ppb | 4.36 | 44.21/49.97 | 23.27/24.84 |
| 0.05 ppb | 4.86 (<5.0) | 72.92/56.59 | 47.75/32.39 |
| 0 | 23.35 | 88.20/40.60 | 87.09/34.97 |

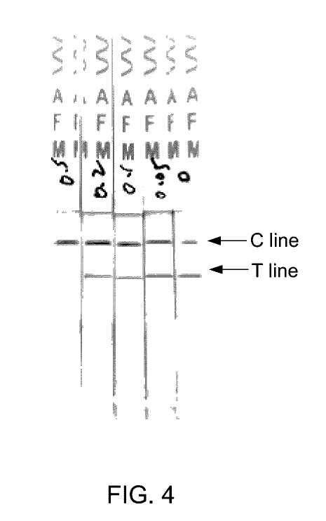

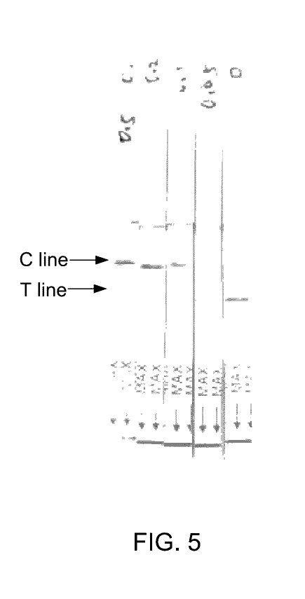

With reference to the experimental data recorded in Table 1, and please simultaneously refer to FIG. 4 and FIG. 5, there are shown image views of multi mono-strip-type aflatoxin kits provided by manufacturer A and manufacturer B, respectively. Through Table 1, FIG. 3, FIG. 4, and FIG. 5, it can find that, the maximum sensitivities of the mono-strip-type aflatoxin kits provided by manufacturer A and manufacturer B for aflatoxin M1 are respectively 0.5 ppb and 0.2 ppb. However, the maximum sensitivity of the immunoassay kit 1 provided by the present invention for aflatoxin M1 is 0.05 ppb. That is, the maximum sensitivity of the immunoassay kit is greater than the commercial mono-strip-type aflatoxin kits by at least 10 folds. In the experiment 1, qualitative analysis result (positive or negative) of the immunochromatographic test carried out by the commercial mono-strip-type aflatoxin kits is determined by naked eyes. However, the naked eyes cannot precisely verify the qualitative analysis result when the C/T value (C value-T value) is smaller than 10. For instance, the C/T value obtained by using the commercial mono-strip-type aflatoxin kits provided by manufacturer A to detect 0.2 ppb aflatoxin M1 is (47.12-45.92)<10, such that the qualitative analysis result is 0.5 ppb after the verification of the naked eyes. Obviously, such verification for the qualitative analysis is incorrect. The similar incorrect verification made by naked eyes also occurs on the commercial mono-strip-type aflatoxin kits provided by manufacturer B. Differing from the commercial mono-strip-type aflatoxin kits, a correct qualitative analysis result (positive or negative) of the immunoassay kit provided by the present invention can be easily obtained by using naked eyes to determine whether the test line 12T shows the color reaction or not. Of course, it can further use an reader to precisely read out the T value of the qualitative analysis carried out by this novel immunoassay kit. For this immunoassay kit, the naked eyes can see the color reaction of the test line 12T when the T value is greater than 5, and that means the qualitative analysis result is negative. On the contrary, when the T value is smaller than 5, the naked eyes cannot see the color reaction of the test line 12T, such that the qualitative analysis result verified to be positive.

Thus, the first embodiment of the immunoassay kit 1 provided by the present invention and the practicability thereof have been described completely and clearly. Subsequently, a second embodiment designed for the immunoassay kit 1 will be further introduced in follows.

the second embodiment of the immunoassay kit 1 is designed for facilitating the immunoassay kit 1 be able to detect the bran wheat included in a test sample. Bran wheat, also known as gluten, gliadin, and gluten protein, is one kind of gluten existing in cereals, such as barley, wheat, oats, rye and so on. According to research and statistics data, a small number of people (˜1%) would be subject to celiac disease after eating the food including gluten. For above reason, U.S. Food and Drug Administration (FDA) especially limits that the concentration of the gluten containing by processed food cannot exceed 20 parts per million (ppm).

### Second Embodiment

The first embodiment of the immunoassay kit 1 is design for carrying out a sandwich-type immunochromatographic test. As shown in FIG. 2, a first antibody (Ab1) conjugating with a marker (CGC-Ab1) is disposed on the conjugation pad 111 of the release strip 11 in the second embodiment of the immunoassay kit 1, wherein the said first antibody is an anti-gliadin antibody. Different from the conjugation pad 111, the control line 12C is formed by a second antibody (which is anti-Ab1) and the test line 12T is formed by an antibody2 (Ab2). Herein, the said second antibody can be goat anti-rabbit immunoglobulin G (IgG), rabbit anti-mouse immunoglobulin G, or goat anti-mouse immunoglobulin G. In addition, the said antibody2 is an anti-gliadin antibody2. The first antibody (Ab1) and the antibody2 (Ab2) could be monoclonal antibody or polyclonal antibody.

Herein, it needs to further explain that, the purpose of the related design for the first embodiment is to make the immunoassay kit 1 detect the gliadin existing in the test sample effectively and rapidly. However, the above described related design for the first embodiment cannot be used for limiting any practicable embodiment of the immunoassay kit 1 proposed by the present invention. The primary technology feature of the present invention is that the immunoassay kit 1 consists of one release strip 11 and one reaction strip 12. So that, based on this technology feature, it reasonably believes that, the person ordinarily skilled in immunoassay kit art can based on their engineering experiences to properly change the kinds of the antibody and/or antigen, so as to facilitate the immunoassay kit 1 be able to detect any one kind of target objective, such as antigen, virus, protein, DNA, RNA, small molecule and so on.

In order to verify the practicability of the second embodiment of the immunoassay kit 1, the inventors of the immunoassay kit 1 has complete related experiments for prove that.

### Experiment 2

### Treating a Sandwich-Type Immunochromatographic Test to a Liquid Dairy Product

Liquid dairy product is also taken as the test sample in experiment 2. After the liquid sample is cooled down to room temperature, 15 drops of the liquid sample are added into a bottle containing extracting solution A by using a first dropper; and then, the bottle is shaken up and down for 30 seconds, so as to evenly mix the liquid sample and the extracting solution A. Subsequently, 3 drops of the extracting solution A are moved from the bottle to small tube containing dilution solution B by using a second dropper; and then the small tube is shaken up and down for 30 seconds, so as to evenly mix the extracting solution A and the dilution solution B to a sample solution. Next, after letting the front end (i.e., the conjugation pad 111) of the release strip 11 be put into the small tube, the small tube is slightly rotated for 30 seconds, so as to make the first antibody (Ab1) (i.e., anti-gliadin antibody) conjugating with the marker (CGC-Ab1) be released into the sample solution in the small tube from conjugation pad 111. Meanwhile, the sample solution would show a specific color, e.g., light pink. After statically standing the small tube for 3 minutes, the gliadin included by the sample solution would connect to the first antibody (Ab1)

Subsequently, after removing the release strip 11 from the small tube, the front end of the reaction strip 12 is put into the small tube, so as to make the sample pad 122 be soaked in the sample solution for 10-15 minutes. Meanwhile, the antibody2 (Ab2) (i.e., anti-gliadin antibody Ab2) fixed on the test line 12T would connect to the gliadin included by the sample solution, and the test line 12T therefore shows a color reaction. Moreover, because the second antibody (anti-Ab1) (i.e., Immunoglobulin G (IgG)) would also connect to the antibody released by the release pad 11, the control line 12C does also show the color reaction. Briefly, when qualitative analysis result of the sandwich type immunochromatographic test is positive, both the test line 12T and the control line 12C would show the color reaction simultaneously. On the contrary, when qualitative analysis result of the sandwich type immunochromatographic test is negative, there is only that the control line 12C would show the color reaction.

Furthermore, a comparison experiment between commercial mono-strip-type gliadin kits and the immunoassay kit of the present invention has been made by the inventors, and related experimental data are therefore recorded in following Table 2.

TABLE 2

| | color verification | color verification |
|---|---|---|
| | value obtained | value obtained |
| | by using | by using |
| | chromatometry | chromatometry |
| | mono-strip-type | the immunoassay |
| concen- | gliadin kits | kit 1 provided by |
| tration | provided by | the present |
| of gliadin | manufacturer C | invention |
| (ppm) | T value | T value |
| 100 | 68.53 | 94.76 |
| 10 | 22.79 | 50.59 |
| 1 | 9.34 | 26.12 |
| 0.1 | 3.83 | 7.96 |
| 0 | 0.04 | 0.11 |

Through Table 2, it can find that, the maximum sensitivities of the mono-strip-type gliadin kits provided by manufacturer C is 1.0 ppm. However, the maximum sensitivity of the immunoassay kit 1 provided by the present invention for gliadin is 0.1 ppm. That is, the maximum sensitivity of the immunoassay kit is greater than the commercial mono-strip-type gliadin kits by at least 10 folds.

Therefore, through above descriptions, the immunoassay kit 1 provided by the present invention have been introduced completely and clearly; in summary, the present invention includes the advantages of:

(1) Differing from conventional lateral flow immunoassay test strips, the present invention provides an immunoassay kit comprising a release strip and a reaction strip. When using this immunoassay kit to complete a competitive-type immunochromatographic test or a sandwich-type immunochromatographic test for a test sample, it needs to firstly put the release strip into the test sample for releasing a specific antibody (or antigen) conjugating with a marker into the test sample, so as to make the first antibody connect to a target object in the test sample; after that, the reaction strip is eventually put into the test sample after the release strip is removed out of the test sample. Therefore, the qualitative analysis result of the competitive/sandwich-type immunochromatographic test can be easily obtained by determining whether the test line shows the color reaction or not.

(2) Inheriting to above point (1), the experimental data obtained from the EXPERIMENT 1 and EXPERIMENT 1 have proved that the maximum sensitivity of the immunoassay kit is greater than the commercial mono-strip-type aflatoxin/gliadin kits by at least 10 folds.

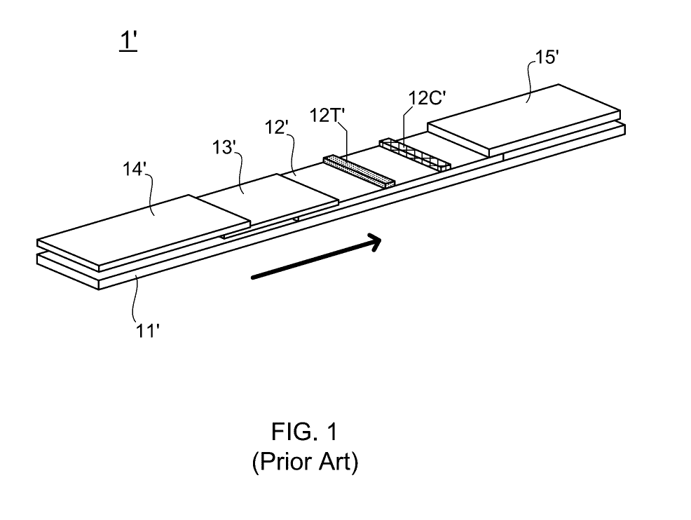

(3) In addition, the conjugation pad the conventional lateral flow immunoassay test strip 1′ shown as FIG. 1 is commonly made of microporous materials with high manufacturing cost and need to store at low temperature (4° C.), but such material's maximum storage life is merely 1 year. Contrary to the conventional lateral flow immunoassay test strip 1′, the manufacturing cost of this novel immunoassay kit is relative low, and the maximum storage life of the immunoassay kit is up to 2 years when being stored in a room-temperature environment.

Herein, it needs to further explain that, if the test sample is urine, the sample buffer will not be used when carrying out the immunochromatographic test. That is, when using this immunoassay kit to complete a competitive-type immunochromatographic test or a sandwich-type immunochromatographic test for a urine sample, the release strip 11 can be directly put into the urine sample for releasing a specific antibody (or antigen) conjugating with a marker into the sample, without using any buffer to dilute the urine in advance.

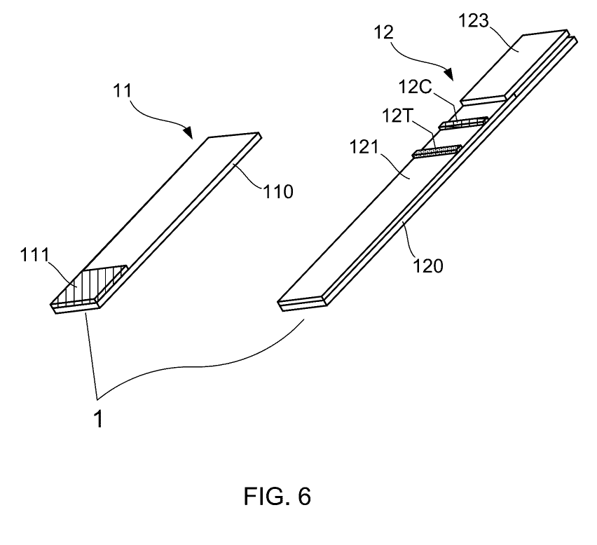

Continuously, please refer to FIG. 6, which shows a schematic structural view of a second exemplary embodiment of the immunoassay kit according to the present invention. The second exemplary embodiment of the immunoassay kit 1 is also consisted of a release strip 11 and a reaction strip 12. However, differing from the first exemplary embodiment of the immunoassay kit 1 shown in FIG. 2, the reaction strip 12 of the second exemplary embodiment does not provided with a sample pad thereon. Instead of that, in the second exemplary embodiment, the membrane 121 on the substrate 120 is extended to the front end of the substrate 120.

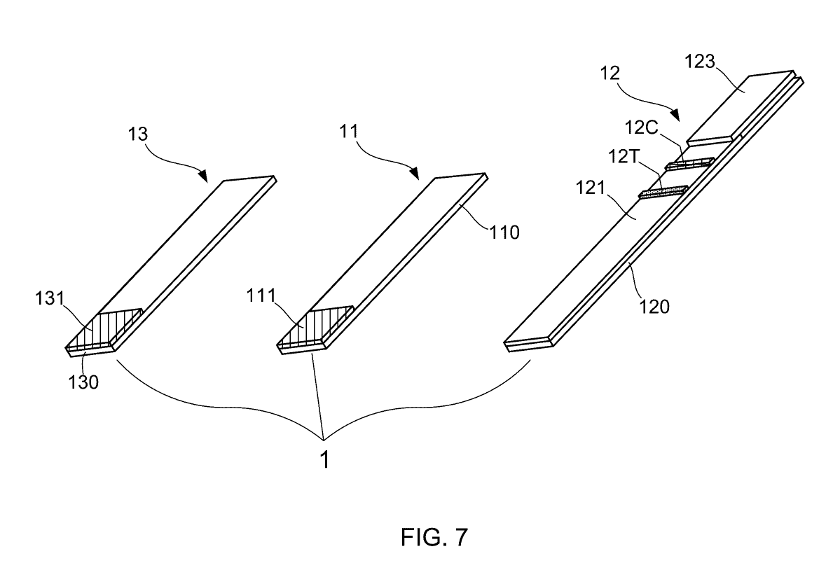

Furthermore, please refer to FIG. 7, which shows a schematic structural view of a third exemplary embodiment of the immunoassay kit according to the present invention. The third exemplary embodiment of the immunoassay kit 1 is also consisted of a release strip 11 and a reaction strip 12. However, differing from the first and second exemplary embodiments of the immunoassay kit 1 shown in FIG. 2 and FIG. 6, the third exemplary embodiment further comprises a pre-process strip 13, consisting of: a substrate 130 and a carrying pad 131. In the pre-process strip 13, the carrying pad 131 is disposed on the substrate 130 and carrying with a pre-processing object, such as ampicillin.

When using this novel immunoassay kit 1 to detect whether a milk sample includes antibiotics enzyme of β-lactam or not, it needs use the pre-process strip 13 for treating a pre-process to the milk sample. To complete the said pre-process, the front end of the pre-process strip 13 is put into the milk sample, so as to release the pre-processing object (i.e., ampicillin) into the milk sample. Next, after the pre-process strip 13 is removed from the milk sample, the front end of the release trip 11 is put into the milk sample for releasing the penicillin enzyme antibody connecting with a maker disposed in the conjugation pad 111 into the milk sample. Meanwhile, when the milk contains the penicillin enzyme antibody, the ampicillin would be hydrolyzed by the penicillin enzyme, such that there has no penicillin enzyme for connecting to the penicillin enzyme antibody. Eventually, after the reaction strip 12 is put into the milk sample, the penicillin enzyme antibody connecting with the maker in the milk sample would connect to the antigen fixed on the test line 12T, so as to make the test line 12T show a color reaction.

The above description is made on embodiments of the present invention. However, the embodiments are not intended to limit scope of the present invention, and all equivalent implementations or alterations within the spirit of the present invention still fall within the scope of the present invention.

What is claimed is:

1. An immunoassay kit, used for detecting a target object from a test sample by way of a competitive-type immunochromatographic test, comprising:
a release strip, being provided with a first antibody conjugating with a marker thereon; and
a reaction strip, being provided with a membrane having a control line and a test line thereon, wherein the control line is formed by a second antibody, and the test line being formed by a conjugate consisting of a specific antigen and a biomolecule;
wherein the first antibody is used for connecting to the target object, and the said specific antigen is the antigen of the target object;
wherein when completing the competitive-type immunochromatographic test by using the immunoassay kit, the release strip being firstly put into the test sample for releasing the first antibody conjugating with the marker into the test sample, and the reaction strip is eventually put into the test sample after the release strip is removed out of the test sample; therefore, the control line on the reaction strip would show a color reaction when the target object is included by the test sample.

2. The immunoassay kit of claim 1, wherein the test sample is selected from the group consisting of: an urine, a tissue fluid, a gravy, a soup, a liquid dairy product, a liquid sample obtained by dissolving a solid sample in a solvent, and an extract of the aforesaid liquid sample.

3. The immunoassay kit of claim 1, wherein the biomolecule is selected from the group consisting of: bovine serum albumin (BSA), ovalbumin (OVA), and choline monooxygenase (CMO).

4. The immunoassay kit of claim 1, wherein the target object is selected from the group consisting of: antigen, virus, protein, DNA, RNA, and small molecule.

5. The immunoassay kit of claim 1, wherein the marker is selected from the group consisting of: colloidal gold, colloidal selenium, latex, nano silver, and nano carbon.

6. The immunoassay kit of claim 1, wherein both the first antibody and the second antibody can be a monoclonal antibody or a polyclonal antibody.

7. The immunoassay kit of claim 1, wherein the release strip comprises:
a substrate; and
a conjugation pad, being made of a microporous material and disposed on the substrate, wherein the first antibody conjugating with the marker thereon is disposed on the conjugation pad.

8. The immunoassay kit of claim 1, wherein the reaction strip comprises:
a substrate, wherein the membrane is disposed on the substrate; and
an absorption pad, being disposed on the substrate and connected to one end of the membrane.

9. The immunoassay kit of claim 2, wherein the aforesaid liquid dairy product is selected from the group consisting of: a raw milk, a sterilized whole milk, a low-fat milk, a drink including dairy compositions, and a derivative product of milk.

10. The immunoassay kit of claim 2, wherein the aforesaid solid sample is a milk powder, a reconcile milk powder, or a feed, and the aforesaid solvent being water or a sample buffer.

11. The immunoassay kit of claim 8, further comprising a pre-process strip, comprising:
a substrate; and
a carrying pad, being disposed on the substrate and carrying with a pre-processing object;
wherein when using the immunoassay kit to detect whether the test sample includes the aforesaid antigen, virus, protein, DNA, RNA, or small molecule or not, the pre-process strip being used for treating a pre-process to the test sample.

12. The immunoassay kit of claim 8, wherein the reaction strip further comprises a sample pad, being disposed on the substrate and connected to the other end of the membrane.

13. The immunoassay kit of claim 8, wherein the manufacturing material of the membrane is selected from the group consisting of: nitrocellulos (NC), polyvinylidene difluoride (PVDF), and nylon; moreover, the manufacturing material of the sample pad is selected from the group consisting of: polyethylene (PE) and glass fiber.

14. The immunoassay kit of claim 10, further comprising a dilution buffer for diluting the test sample, wherein the dilution buffer is a phosphate solution; moreover, the aforesaid sample buffer can also be the phosphate solution or a high-concentration phosphate solution.

15. An immunoassay kit, used for detecting a target object from a test sample by way of a sandwich-type immunochromatographic test, comprising:
a release strip, being provided with a first antibody conjugating with a marker thereon, wherein the first antibody is used for connecting to the target object; and
a reaction strip, being provided with a membrane having a control line and a test line thereon, wherein the control line is formed by second antibody, and the test line being formed by an antibody2; moreover, the first antibody and the antibody2 are used for connecting to the target object;
wherein when completing the sandwich-type immunochromatographic test by using the immunoassay kit, the release strip being firstly put into the test sample for releasing the first antibody conjugating with the marker into the test sample, and the reaction strip is eventually put into the test sample after the release strip is removed out of the test sample; therefore, the control line and the test line on the reaction strip would simultaneously show a color reaction when the target object is included by the test sample.

16. The immunoassay kit of claim 15, wherein the test sample is selected from the group consisting of: an urine, a tissue fluid, a gravy, a soup, a liquid dairy product, a liquid sample obtained by dissolving a solid sample in a solvent, and an extract of the aforesaid liquid sample.

17. The immunoassay kit of claim 15, wherein the marker is selected from the group consisting of: colloidal gold, colloidal selenium, latex, nano silver, and nano carbon.

18. The immunoassay kit of claim 15, wherein all the first antibody, and the antibody2 can be a monoclonal antibody or a polyclonal antibody.

19. The immunoassay kit of claim 15, wherein the target object is selected from the group consisting of: antigen, virus, protein, DNA, RNA, and small molecule.

20. The immunoassay kit of claim 15, wherein the release strip comprises:
a substrate; and
a conjugation pad, being made of a microporous material and disposed on the substrate, wherein the first antibody conjugating with the marker thereon is disposed on the conjugation pad.

21. The immunoassay kit of claim 15, wherein the reaction strip comprises:
a substrate, wherein the membrane is disposed on the substrate; and
an absorption pad, being disposed on the substrate and connected to one end of the membrane.

22. The immunoassay kit of claim 16, wherein the aforesaid liquid dairy product is selected from the group consisting of: a raw milk, a sterilized whole milk, a low-fat milk, a drink including dairy compositions, and a derivative product of milk.

23. The immunoassay kit of claim 16, wherein the aforesaid solid sample is a milk powder, a reconcile milk powder, or a feed, and the aforesaid solvent being water or a sample buffer.

24. The immunoassay kit of claim 19, further comprising a pre-process strip, comprising:
a substrate; and
a carrying pad, being disposed on the substrate and carrying with a pre-processing object;
wherein when using the immunoassay kit to detect whether the test sample includes the aforesaid antigen, virus, protein, DNA, RNA, or small molecule or not, the pre-process strip being used for treating a pre-process to the test sample.

25. The immunoassay kit of claim 21, wherein the manufacturing material of the membrane can be nitrocellulos (NC), polyvinylidene difluoride (PVDF), and nylon; moreover, the manufacturing material of the sample pad is selected from the group consisting of: polyethylene (PE) or glass fiber.

26. The immunoassay kit of claim 21, wherein the reaction strip further comprises a sample pad, being disposed on the substrate and connected to the other end of the membrane.

27. The immunoassay kit of claim 23, further comprising a dilution buffer for diluting the test sample, wherein the dilution buffer is a phosphate solution; moreover, the aforesaid sample buffer can also be the phosphate solution or a high-concentration phosphate solution.
